(19) United States
(12) Patent Application Publication
(10) Pub. No.: US 2017/0368069 A1
(43) Pub. Date: Dec. 28, 2017

(54) USE OF 4-(4-FLUORO-2-METHOXYPHENYL)-N--1,3,5-TRIAZIN-2-AMINE FOR TREATING LEUKEMIAS

(71) Applicant: Bayer Pharma Aktiengesellschaft, Berlin (DE)

(72) Inventor: Arne SCHOLZ, Berlin (DE)

(73) Assignee: Bayer Pharma Aktiengesellschaft, Berlin (DE)

(21) Appl. No.: 15/543,228

(22) PCT Filed: Jan. 8, 2016
(86) PCT No.: PCT/EP2016/050251

§ 371 Date: Jul. 12, 2017

(30) Foreign Application Priority Data
Jan. 13, 2015 (EP) 15150940.3

(51) Int. Cl. A61K 31/53; C12Q 1/68

(52) U.S. Cl. CPC A61K 31/53; C12Q 1/6886; C12Q 2600/156; C12Q 2600/106

(57) ABSTRACT

The present invention relates to the use of 4-(4-Fluoro-2-methoxyphenyl)-N-{3-[(S-methylsulfonimidoyl)methyl]phenyl}-1,3,5-triazin-2-amine (compound A), more particularly (+)-4-(4-Fluoro-2-methoxyphenyl)-N-{3-[(S-methylsulfonimidoyl)methyl]phenyl}-1,3,5-triazin-2-amine (compound A′), for treating leukemias, in particular acute leukemias, preferably acute myeloid leukemias.

The present invention relates to the use of 4-(4-Fluoro-2-methoxyphenyl)-N-{3-[(S-methylsulfonimidoyl)methyl]phenyl}-1,3,5-triazin-2-amine (compound A), more particularly (+)-4-(4-Fluoro-2-methoxyphenyl)-N-{3-[(S-methylsulfonimidoyl)methyl]phenyl}-1,3,5-triazin-2-amine (compound A′), for treating leukemias, in particular acute leukemias, preferably acute myeloid leukemias.

The family of cyclin-dependent kinase (CDK) proteins consists of members that are key regulators of the cell division cycle (cell cycle CDK's), that are involved in regulation of gene transcription (transcriptional CDK's), and of members with other functions. CDKs require for activation the association with a regulatory cyclin subunit. The cell cycle CDKs CDK1/cyclin B, CDK2/cyclin A, CDK2/cyclinE, CDK4/cyclinD, and CDK6/cyclinD get activated in a sequential order to drive a cell into and through the cell division cycle. The transcriptional CDKs CDK9/cyclin T and CDK7/cyclin H regulate the activity of RNA polymerase II via phosphorylation of the carboxy-terminal domain (CTD). Positive transcription factor b (P-TEFb) is a heterodimer of CDK9 and one of four cyclin partners, cyclin T1, cyclin K, cyclin T2a or T2b.

Whereas CDK9 (NCBI GenBank Gene ID 1025) is exclusively involved in transcriptional regulation, CDK7 in addition participates in cell cycle regulation as CDK-activating kinase (CAK).

Transcription of genes by RNA polymerase II is initiated by assembly of the pre-initiation complex at the promoter region and phosphorylation of Ser 5 and Ser 7 of the CTD by CDK7/cyclin H. For a major fraction of genes RNA polymerase II stops mRNA transcription after it moved 20-40 nucleotides along the DNA template. This promoter-proximal pausing of RNA polymerase II is mediated by negative elongation factors and is recognized as a major control mechanism to regulate expression of rapidly induced genes in response to a variety of stimuli (Cho et al., Cell Cycle 2010, 9, 1697). P-TEFb is crucially involved in overcoming promoter-proximal pausing of RNA polymerase II and transition into a productive elongation state by phosphorylation of Ser 2 of the CTD as well as by phosphorylation and inactivation of negative elongation factors.

Activity of P-TEFb itself is regulated by several mechanisms. About half of cellular P-TEFb exists in an inactive complex with 7SK small nuclear RNA (7SK snRNA), La-related protein 7 (LARP7/PIP7S) and hexamethylene bis-acetamide inducible proteins 1/2 (HEXIM1/2, He et al., Mol. Cell 2008, 29, 588). The remaining half of P-TEFb exists in an active complex containing the bromodomain protein Brd4 (Yang et al., Mol. Cell 2005, 19, 535). Brd4 recruits P-TEFb through interaction with acetylated histones to chromatin areas primed for gene transcription. Through alternately interacting with its positive and negative regulators, P-TEFb is maintained in a functional equilibrium: P-TEFb bound to the 7SK snRNA complex represents a reservoir from which active P-TEFb can be released on demand of cellular transcription and cell proliferation (Zhou & Yik, Microbiol. Mol. Biol. Rev. 2006, 70, 646). Furthermore, the activity of P-TEFb is regulated by posttranslational modifications including phosphorylation/de-phosphorylation, ubiquitination, and acteylation (reviewed in Cho et al., Cell Cycle 2010, 9, 1697).

Deregulated activity of CDK9 kinase activity of the P-TEFb heterodimer is associated with a variety of human pathological settings such as hyper-proliferative diseases (e.g. cancer), virally induced infectious diseases or cardiovascular diseases.

Cancer is regarded as a hyper-proliferative disorder mediated by a disbalance of proliferation and cell death (apoptosis). High levels of anti-apoptotic Bcl-2-family proteins are found in various human tumors and account for prolonged survival of tumor cells and therapy resistance. Inhibition of P-TEFb kinase activity was shown to reduce transcriptional activity of RNA polymerase II leading to a decline of short-lived anti-apoptotic proteins, especially Mcl-1 and XIAP, reinstalling the ability of tumor cells to undergo apoptosis. A number of other proteins associated with the transformed tumor phenotype (such as Myc, NF-kB responsive gene transcripts, mitotic kinases) are either short-lived proteins or are encoded by short-lived transcripts which are sensitive to reduced RNA polymerase II activity mediated by P-TEFb inhibition (reviewed in Wang & Fischer, Trends Pharmacol. Sci. 2008, 29, 302).

Many viruses rely on the transcriptional machinery of the host cell for the transcription of their own genome. In case of HIV-1 RNA polymerase II gets recruited to the promoter region within the viral LTR's. The viral transcription activator (Tat) protein binds to nascent viral transcripts and overcomes promoter-proximal RNA polymerase II pausing by recruitment of P-TEFb which in turn promotes transcriptional elongation. Furthermore, the Tat protein increases the fraction of active P-TEFb by replacement of the P-TEFb inhibitory proteins HEXIM1/2 within the 7SK snRNA complex. Recent data have shown that inhibition of the kinase activity of P-TEFb is sufficient to block HIV-1 replication at kinase inhibitor concentrations that are not cytotoxic to the host cells (reviewed in Wang & Fischer, Trends Pharmacol. Sci. 2008, 29, 302). Similarly, recruitment of P-TEFb by viral proteins has been reported for other viruses such as B-cell cancer-associated Epstein-Barr virus, where the nuclear antigen EBNA2 protein interacts with P-TEFb (Bark-Jones et al., Oncogene 2006, 25, 1775), and the human T-lymphotropic virus type 1 (HTLV-1), where the transcriptional activator Tax recruits P-TEFb (Zhou et al., J. Virol. 2006, 80, 4781).

Cardiac hypertrophy, the heart's adaptive response to mechanical overload and pressure (hemodynamic stress e.g. hypertension, myocardial infarction), can lead, on a long term, to heart failure and death. Cardiac hypertrophy was shown to be associated with increased transcriptional activity and RNA polymerase II CTD phosphorylation in cardiac muscle cells. P-TEFb was found to be activated by dissociation from the inactive 7SK snRNA/HEXIM1/2 complex. These findings suggest pharmacological inhibition of P-TEFb kinase activity as a therapeutic approach to treat cardiac hypertrophy (reviewed in Dey et al., Cell Cycle 2007, 6, 1856).

In summary, multiple lines of evidence suggest that selective inhibition of the CDK9 kinase activity of the P-TEFb heterodimer (=CDK9 and one of four cyclin partners, cyclin T1, cyclin K, cyclin T2a or T2b) represents an innovative approach for the treatment of diseases such as cancer, viral diseases, and/or diseases of the heart. CDK9 belongs to a family of at least 13 closely related kinases of which the subgroup of the cell cycle CDK's fulfills multiple roles in regulation of cell proliferation. Thus, co-inhibition of cell cycle CDK's (e.g. CDK1/cyclin B, CDK2/cyclin A, CDK2/cyclinE, CDK4/cyclinD, CDK6/cyclinD) and of CDK9 is expected to impact normal proliferating tissues such as intestinal mucosa, lymphatic and hematopoietic organs, and reproductive organs. To maximize the therapeutic margin of CDK9 kinase inhibitors, molecules with high selectivity towards CDK9 are therefore required.

CDK inhibitors in general as well as CDK9 inhibitors are described in a number of different publications: WO2008129070 and WO2008129071 both describe 2,4 disubstituted aminopyrimidines as CDK inhibitors in general. It is also asserted that some of these compounds may act as selective CDK9 inhibitors (WO2008129070) and as CDK5 inhibitors (WO2008129071), respectively, but no specific CDK9 IC50 (WO2008129070) or CDK5 IC50 (WO200812971) data is presented.

WO2008129080 discloses 4,6 disubstituted aminopyrimidines and demonstrates that these compounds show an inhibitory effect on the protein kinase activity of various protein kinases, such as CDK1, CDK2, CDK4, CDK5, CDK6 and CDK9, with a preference for CDK9 inhibition (example 80). EP1218360 B1 describes triazin derivatives as kinase inhibitors, but does not disclose potent or selective CDK9 inhibitors.

WO2008079933 discloses aminopyridine and aminopyrimidine derivatives and their use as CDK1, CDK2, CDK3, CDK4, CDK5, CDK6, CDK7, CDK8 or CDK9 inhibitors.

WO2011012661 describes aminopyridine derivatives useful as CDK inhibitors.

Wang et al. (Chemistry & Biology 2010, 17, 1111-1121) describe 2-anilino-4-(thiazol-5-yl)pyrimidine transcriptional CDK inhibitors, which show anticancer activity in animal models.

WO2004009562 discloses substituted triazine kinase inhibitors. For selected compounds CDK1 and CDK 4 test data, but no CDK9 data is presented.

WO2004072063 describes heteroaryl (pyrimidine, triazine) substituted pyrroles as inhibitors of protein kinases such as ERK2, GSK3, PKA or CDK2.

WO2010009155 discloses triazine and pyrimidine derivatives as inhibitors of histone deacetylase and/or cyclin dependent kinases (CDKs). For selected compounds CDK2 test data is described.

WO2003037346 (corresponding to U.S. Pat. No. 7,618,968B2, U.S. Pat. No. 7,291,616B2, US2008064700A1, US2003153570A1) relates to aryl triazines and uses thereof, including to inhibit lysophosphatidic acid acyltransferase beta (LPAAT-beta) activity and/or proliferation of cells such as tumor cells.

WO2008025556 describes carbamoyl sulfoximides having a pyrimidine core, which are useful as kinase inhibitors. No CDK9 data is presented.

WO2002066481 describes pyrimidine derivatives as cyclin dependent kinase inhibitors CDK9 is not mentioned and no CDK9 data is presented.

WO2008109943 concerns phenyl aminopyri(mi)dine compounds and their use as kinase inhibitors, in particular as JAK2 kinase inhibitors. The specific examples focus on compounds having a pyrimidine core.

WO2009032861 describes substituted pyrimidinyl amines as JNK kinase inhibitors. The specific examples focus on compounds having a pyrimidine core.

WO2011046970 concerns amino-pyrimidine compounds as inhibitors of TBKL and/or IKK epsilon. The specific examples focus on compounds having a pyrimidine core.

WO2012160034 the compounds of the present invention. It is disclosed the compounds inhibit the cell proliferation of HeLa cells (cervical cancer), HeLa/MaTu/ADR cells (cervical cancer), NCI-H460 cells (non-small cell lung cancer), DU145 cells (hormone-independent human prostate cancer), Caco-2 cells (colorectal cancer) and B16F10 cells (melanoma).

The object of the present invention is to improve the treatment of acute leukemias, especially acute myeloid leukemias (AML) and to avoid unnecessary treatments by identifying patients prior the treatment who will probably benefit from the treatment.

### Treatment of AML

Therapy for AML includes remission induction followed by postremission chemotherapy for most patients. For some, this is followed by hematopoietic stem cell transplantation. Treatment recommendations for AML vary, taking into account patient age, cytogenetics, and prognostic factors.

The goal of induction chemotherapy is to reduce the number of leukemic cells, as well as to return proper function to the bone marrow. The 7+3 regimen of cytarabine plus an anthracycline or anthracenedione is the most common induction regimen. Potential toxicities of induction therapy include: tumor lysis syndrome, cardiac abnormalities, tissue necrosis, pancytopenia, nausea and vomiting, alopecia, and death, among others. Commonly, a bone marrow biopsy will be repeated 2 weeks following the initiation of therapy, to assess marrow aplasia. If residual leukemia is detected, patients are treated with another chemotherapy course, termed reinduction.

Postremission chemotherapy then aims to eradicate any residual disease in an attempt at cure. Postremission chemotherapy includes high-dose cytarabine (ara-c; HiDAC) for patients younger than 60 years, in whom a survival advantage has been demonstrated with this therapy. In patients younger than 60 years, HiDAC yields a 4-year disease-free survival rate of 44%, but carries with it a 5% treatment-related mortality (Estey E H. Acute myeloid leukemia: 2012 update on diagnosis, risk stratification, and management. Am J Hematol 2012; 87:89-99). High doses of cytarabine can be associated with cerebellar, ophthalmologic, and gastrointestinal toxicity, particularly in patients over the age of 60 years. The treatment of older AML patients is controversial. Older adults often cannot tolerate the toxicities of intensive remission induction chemotherapy. With the typical treatment plans, the treatment-related mortality is between 15% and 30%. Other less intensive regimens may therefore be used. The 5-year disease-free survival rate in these patients is still only 5-10%.

Given this low treatment efficacy and the high rate of treatment related morbidity and mortality there is a high unmet medical need for new treatment modalities in AML

### 11q23 Translocation in AML

(also referred to by other terms including 11q23 rearrangement, 11q rearrangements and the MLL rearrangement)

Cytogenetic analysis of metaphase cells is a key component to the evaluation of all patients with newly diagnosed or suspected acute myeloid leukemia (AML). The malignant cells in most patients with AML have non-random, acquired clonal chromosomal abnormalities. In some cases, specific cytogenetic abnormalities are closely, and sometimes uniquely, associated with morphologically and clinically distinct subsets of the disease. As such, the 2008 WHO classification of tumors of the hematopoietic and lymphoid tissues uses genetic findings in addition to morphologic, immunophenotypic, and clinical features to define distinct subtypes of AML. In addition to establishing the type of AML, specific cytogenetic abnormalities have diagnostic, prognostic, and therapeutic importance.

Using standard banding techniques, 50 to 60 percent of patients with AML de novo have abnormal karyotypes. 5-10% of patients show 11q rearrangements (also referred to by other terms including 11q23 translocations). In these patients the mixed lineage leukemia (MLL) gene (referred to by other names including HRX, ALL-1, Htrx, and KMT2A), located at chromosome band 11q23, is translocated to various different partner loci. MLL encodes a histone methyltransferase that plays a critical role in normal embryonic development and hematopoiesis, mainly regulating transcription via epigenetic mechanisms. Translocations involving MLL result in MLL fusion proteins that appear to deregulate the RNA polymerase II elongation factor thereby allowing for transcriptional elongation free of the usual checkpoints [Mohan M, Lin C, Guest E, Shilatifard A. Licensed to elongate: a molecular mechanism for MLL-based leukaemogenesis. Nat Rev Cancer 2010; 10:721; Lin C, Smith E R, Takahashi H, et al. AFF4, a component of the ELL/P-TEFb elongation complex and a shared subunit of MLL chimeras, can link transcription elongation to leukemia. Mol Cell 2010; 37:429].

Thus, it is crucial to test for cytogenetics and, when applicable, to use fluorescence in situ hybridization, because these may help dictate therapy.

Cytogentic analysis is a standard procedure and i.a. described in
Heim S, Mitelman F. Cancer Cytogenetics, 3rd ed, Wiley, Hoboken, N.J. 2008.
Godley L A, LeBeau M M. Cytogenetics and molecular abnormalities. In: Williams Hematology, 8th ed, Kaushansky K, Lichtman M A, Beutler E, et al. (Eds), McGraw-Hill, Burr Ridge, Ill. 2010.
Swerdlow S H, Campo E, Harris N L, et al. (Eds). World Health Organization Classification of Tumours of Haematopoietic and Lymphoid Tissues, IARC Press, Lyon 2008.

A 11q23 translocation or the resulting MLL-fusion protein can also be detected by other various methods e.g Southern Blot, DNA-PCR, DNA-probes, FISH, RT-PCR, mRNA-probes, Western blot, FACS or ELISA.

It has now been found that the compound 4-(4-Fluoro-2-methoxyphenyl)-N-{3-[(S-methylsulfonimidoyl)methyl]phenyl}-1,3,5-triazin-2-amine (compound A, formula (I)),

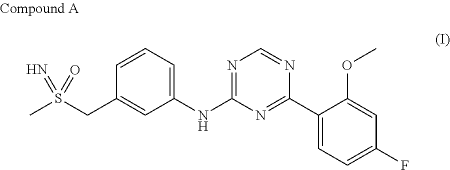 [SMILES: COc1cc(F)ccc1-c1ncnc(Nc2cccc(CS(C)(=N)=O)c2)n1]

more particularly
(+)-4-(4-Fluoro-2-methoxyphenyl)-N-{3-[(S-methylsulfonimidoyl)methyl]phenyl}-1,3,5-triazin-2-amine (compound A′),
   acts in specific tumour types which had previously not yet been contemplated, viz. in leukemias, in particular acute leukemias, preferably acute myeloid leukemias (AML) and more preferably in acute myeloid leukemias with a 11q23 translocation.
4-(4-Fluoro-2-methoxyphenyl)-N-{3-[(S-methylsulfonimidoyl)methyl]phenyl}-1,3,5-triazin-2-amine (compound A) is a selected sulphoximine-substituted anilinopyrimidine derivative which can be separated into two stereoisomers, viz.:
(+)-4-(4-Fluoro-2-methoxyphenyl)-N-{3-[(S-methylsulfonimidoyl)methyl]phenyl}-1,3,5-triazin-2-amine (compound A′) and
(−)-4-(4-Fluoro-2-methoxyphenyl)-N-{3-[(S-methylsulfonimidoyl)methyl]phenyl}-1,3,5-triazin-2-amine (compound A″).

Compound A′ is preferred and in clinical development as BAY1143572.

Where compound A is mentioned below, both the pure stereoisomers A′ and A″, and also any mixture of these two, are meant thereby.

The present invention is directed to the use of
4-(4-Fluoro-2-methoxyphenyl)-N-{3-[(S-methylsulfonimidoyl)methyl]phenyl}-1,3,5-triazin-2-amine (compound A),
   more particularly
(+)-4-(4-Fluoro-2-methoxyphenyl)-N-{3-[(S-methylsulfonimidoyl)methyl]phenyl}-1,3,5-triazin-2-amine (compound A′),
   for the treatment and/or prophylaxis of leukemias, in particular acute leukemias, preferably acute myeloid leukemias (AML) and more preferably acute myeloid leukemias with a 11q23 translocation.

Preferred is the use of compound A, preferably compound A′, for the treatment and/or prophylaxis of acute myeloid leukemia, wherein the subject who shall be treated is one for whom a 11q23 translocation has been detected in a tissue sample containing tumor cells from the subject. The translocation can be detected by a cytogenetic analysis.

The present application is further directed to the use of
4-(4-Fluoro-2-methoxyphenyl)-N-{3-[(S-methylsulfonimidoyl)methyl]phenyl}-1,3,5-triazin-2-amine,
   more particularly
(+)-4-(4-Fluoro-2-methoxyphenyl)-N-{3-[(S-methylsulfonimidoyl)methyl]phenyl}-1,3,5-triazin-2-amine,
   for preparing a medicament for treating leukemias, in particular acute leukemias, preferably acute myeloid leukemias (AML) and more preferably acute myeloid leukemias with a 11q23 translocation.

Another aspect of the present invention is the use of 4-(4-Fluoro-2-methoxyphenyl)-N-{3-[(S-methylsulfonimidoyl)methyl]phenyl}-1,3,5-triazin-2-amine (compound A) according to formula (I)

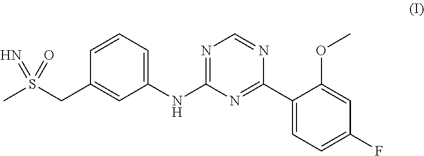 [SMILES: COc1cc(F)ccc1-c1ncnc(Nc2cccc(CS(C)(=N)=O)c2)n1]

in the manufacture of a medicament for treating cancer in a subject, wherein the medicament is manufactured for treating leukemias.

The use of enantiomer
(+)-4-(4-Fluoro-2-methoxyphenyl)-N-{3-[(S-methylsulfonimidoyl)methyl]phenyl}-1,3,5-triazin-2-amine (compound A′) in the manufacture of a medicament for treating leukemias is preferred.

Preferred is the use of compound A or compound A′ in the manufacture of a medicament for treating acute leukemias, preferably for treating acute myeloid leukemias and more preferably acute myeloid leukemias with a 11q23 translocation.

Especially preferred is the use of compound A′ in the manufacture of a medicament for treating acute myeloid leukemia wherein the subject who shall be treated is one for whom a 11q23 translocation has been detected in a tissue sample containing tumor cells from the subject. The translocation can be detected by a cytogenetic analysis.

Another aspect of the present invention is the use of a compound A or one of its physiologically acceptable salts, diastereomers or enantiomers in the manufacture of a medicament for treating acute myeloid leukemia in a subject wherein a 11q23 translocation is determined in tumour tissue or in tumour cells of the subject.

The present application further provides 4-(4-Fluoro-2-methoxyphenyl)-N-{3-[(S-methylsulfonimidoyl)methyl]phenyl}-1,3,5-triazin-2-amine,

more particularly
(+)-4-(4-Fluoro-2-methoxyphenyl)-N-{3-[(S-methylsulfonimidoyl)methyl]phenyl}-1,3,5-triazin-2-amine,
   for treating leukemias, in particular acute leukemias, preferably acute myeloid leukemias (AML) and more preferably acute myeloid leukemias with a 11q23 translocation.

The present invention is also directed to
4-(4-Fluoro-2-methoxyphenyl)-N-{3-[(S-methylsulfonimidoyl)methyl]phenyl}-1,3,5-triazin-2-amine according to formula I (compound A)

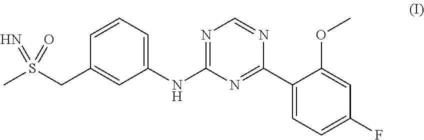 [SMILES: COc1cc(F)ccc1-c1ncnc(Nc2cccc(CS(C)(=N)=O)c2)n1]

for treating leukemias, in particular acute leukemias, preferably acute myeloid leukemias (AML) and more preferably acute myeloid leukemias with a 11q23 translocation.

The present invention is preferred directed to
(+)-4-(4-Fluoro-2-methoxyphenyl)-N-{3-[(S-methylsulfonimidoyl)methyl]phenyl}-1,3,5-triazin-2-amine (compound A′) for treating leukemias, in particular acute leukemias, preferably acute myeloid leukemias (AML) and more preferably acute myeloid leukemias with a 11q23 translocation.

Another aspect of the present invention is compound A, preferably compound A′, for treating acute myeloid leukemia, wherein the subject who shall be treated is one for whom a 11q23 translocation has been detected in a tissue sample containing tumor cells from the subject. The translocation can be detected by a cytogenetic analysis.

The present invention is also directed to
4-(4-Fluoro-2-methoxyphenyl)-N-{3-[(S-methylsulfonimidoyl)methyl]phenyl}-1,3,5-triazin-2-amine according to formula I (compound A)

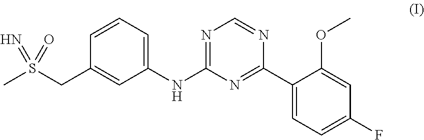 [SMILES: COc1cc(F)ccc1-c1ncnc(Nc2cccc(CS(C)(=N)=O)c2)n1]

for the use in a method of treatment and/or prophylaxis of leukemias, in particular acute leukemias, preferably acute myeloid leukemias (AML) and more preferably acute myeloid leukemias with a 11q23 translocation.

The present invention is preferred directed to
(+)-4-(4-Fluoro-2-methoxyphenyl)-N-{3-[(S-methylsulfonimidoyl)methyl]phenyl}-1,3,5-triazin-2-amine (compound A′) for the use in a method of treatment and/or prophylaxis of leukemias, in particular acute leukemias, preferably acute myeloid leukemias (AML) and more preferably acute myeloid leukemias with a 11q23 translocation.

Another aspect of the present invention is compound A, preferably compound A′, for the use in a method of treatment and/or prophylaxis of acute myeloid leukemia, wherein the subject who shall be treated is one for whom a 11q23 translocation has been detected in a tissue sample containing tumor cells from the subject. The translocation can be detected by a cytogenetic analysis.

Another aspect of the present invention is a

method of treating acute myeloid leukemia comprising the steps
a) assaying a tumor sample from the patient and
b) determining if 11q23 is translocated and
c) administering a therapeutically effective amount of 4-(4-Fluoro-2-methoxyphenyl)-N-{3-[(S-methylsulfonimidoyl)methyl]phenyl}-1,3,5-triazin-2-amine (compound A) according to formula I,
   preferably (+)-4-(4-Fluoro-2-methoxyphenyl)-N-{3-[(S-methylsulfonimidoyl)methyl]phenyl}-1,3,5-triazin-2-amine (compound A′)
   if 11q23 is translocated as defined in step b.

Another aspect of the present invention is a method of treatment and/or prophylaxis of leukemias, in particular acute leukemias, preferably acute myeloid leukemias (AML) and more preferably acute myeloid leukemias with a 11q23 translocation using an effective amount of 4-(4-Fluoro-2-methoxyphenyl)-N-{3-[(S-methylsulfonimidoyl)methyl]phenyl}-1,3,5-triazin-2-amine (compound A) according to formula I

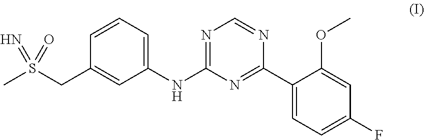 [SMILES: COc1cc(F)ccc1-c1ncnc(Nc2cccc(CS(C)(=N)=O)c2)n1]

preferably
(+)-4-(4-Fluoro-2-methoxyphenyl)-N-{3-[(S-methylsulfonimidoyl)methyl]phenyl}-1,3,5-triazin-2-amine (compound A′).

A preferred method of treatment is a method of treatment and/or prophylaxis of acute myeloid leukemia using an effective amount of compound A, preferably compound A′, wherein the subject who shall be treated is one for whom a 11q23 translocation has been detected in a tissue sample containing tumor cells from the subject. The translocation can be detected by a cytogenetic analysis.

The present application further provides pharmaceutical compositions containing
4-(4-Fluoro-2-methoxyphenyl)-N-{3-[(S-methylsulfonimidoyl)methyl]phenyl}-1,3,5-triazin-2-amine,
   more particularly
(+)-4-(4-Fluoro-2-methoxyphenyl)-N-{3-[(S-methylsulfonimidoyl)methyl]phenyl}-1,3,5-triazin-2-amine,
   for treating leukemias, in particular acute leukemias, preferably acute myeloid leukemias (AML) and more preferably acute myeloid leukemias with a 11q23 translocation.

The present invention is also directed to

pharmaceutical compositions comprising
4-(4-Fluoro-2-methoxyphenyl)-N-{3-[(S-methylsulfonimidoyl)methyl]phenyl}-1,3,5-triazin-2-amine (compound A) according to formula I

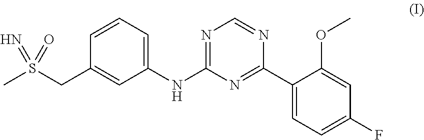 [SMILES: COc1cc(F)ccc1-c1ncnc(Nc2cccc(CS(C)(=N)=O)c2)n1]

preferably
(+)-4-(4-Fluoro-2-methoxyphenyl)-N-{3-[(S-methylsulfonimidoyl)methyl]phenyl}-1,3,5-triazin-2-amine (compound A′)
   in combination with an inert, nontoxic, pharmaceutically suitable adjuvant for the treatment and/or prophylaxis of leukemias, in particular acute leukemias, preferably acute myeloid leukemias (AML) and more preferably acute myeloid leukemias with a 11q23 translocation.

A preferred pharmaceutical composition is a pharmaceutical composition comprising compound A, preferably compound A′, for the treatment of acute myeloid leukemia wherein the subject who shall be treated is one for whom a 11q23 translocation has been detected in a tissue sample containing tumor cells from the subject. The translocation can be detected by a cytogenetic analysis. The translocation can be detected by a cytogenetic analysis.

The present application further provides combinations of
4-(4-Fluoro-2-methoxyphenyl)-N-{3-[(S-methylsulfonimidoyl)methyl]phenyl}-1,3,5-triazin-2-amine (compound A),
   more particularly
(+)-4-(4-Fluoro-2-methoxyphenyl)-N-{3-[(S-methylsulfonimidoyl)methyl]phenyl}-1,3,5-triazin-2-amine (compound A′),
   with at least one further active ingredient for treating leukemias, in particular acute leukemias, preferably acute myeloid leukemias (AML) and more preferably acute myeloid leukemias with a 11q23 translocation.

The present invention is also directed to

pharmaceutical combinations comprising
4-(4-Fluoro-2-methoxyphenyl)-N-{3-[(S-methylsulfonimidoyl)methyl]phenyl}-1,3,5-triazin-2-amine (compound A) according to formula I

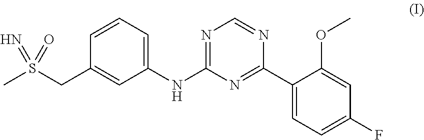 [SMILES: COc1cc(F)ccc1-c1ncnc(Nc2cccc(CS(C)(=N)=O)c2)n1]

preferably
(+)-4-(4-Fluoro-2-methoxyphenyl)-N-{3-[(S-methylsulfonimidoyl)methyl]phenyl}-1,3,5-triazin-2-amine (compound A′)
   in combination with at least one or more further active ingredients for the treatment and/or prophylaxis of leukemias, in particular acute leukemias, preferably acute myeloid leukemias (AML) and more preferably acute myeloid leukemias with a 11q23 translocation.

A preferred pharmaceutical combination is a pharmaceutical combination comprising compound A, preferably compound A′, for the treatment of acute myeloid leukemia wherein the subject who shall be treated is one for whom a 11q23 translocation has been detected in a tissue sample containing tumor cells from the subject. The translocation can be detected by a cytogenetic analysis. The translocation can be detected by a cytogenetic analysis.

Another aspect of the present invention is a method for identifying a patient disposed to respond favorably to a CDK9-inhibitor for treating acute myeloid leukemia,
wherein the CDK9-inhibitor is compound A and
wherein the method comprises the detection of a 11q23 translocation in tumor cells in a tissue sample from the patient.

Preferred is a method for identifying a patient disposed to respond favorably to a CDK9-inhibitor for treating acute myeloid leukemia,
wherein the CDK9-inhibitor is compound A′ and
wherein the method comprises the detection of a 11q23 translocation in tumor cells in a tissue sample from the patient.
   The translocation can be detected by a cytogenetic analysis.

The use of the physiologically tolerable salts of compound A should likewise be considered to be covered by the present invention.

Physiologically safe salts of compound A encompass acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

Physiologically safe salts of compound A also encompass salts of customary bases, such as, by way of example and preferably, alkali metal salts (e.g. sodium and potassium salts), alkaline earth metal salts (e.g. calcium and magnesium salts) and ammonium salts derived from ammonia or organic amines having from 1 to 16 C atoms, such as, by way of example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

The present invention further provides drugs containing compound A and at least one or more further active ingredients for treating leukemias, especially acute myeloid leukemias (AML) and in particular acute myeloid leukemias with a 11q23 translocation.

Compound A may have systemic and/or local activity. For this purpose, it can be administered in a suitable manner, such as, for example, orally, parenterally, via the pulmonary route, nasal, sublingually, lingually, buccally, rectally, vaginally, dermally, transdermally, conjuntivally, otically or as an implant or stent.

For these administration routes, compound A according to the invention may be administered in suitable administration forms.

Suitable for oral administration forms which function according to the prior art and deliver compound A of the invention rapidly and/or in a modified manner and which comprise compound A according to the invention in crystalline and/or amorphized and/or dissolved form, such as, for example, tablets (uncoated or coated tablets, for example with coatings which are resistant to gastric juice or dissolve with a delay or are insoluble and control the release of the compound of the invention), tablets which disintegrate rapidly in the oral cavity, or films/wafers, films/lyophylisates, capsules (for example hard or soft gelatine capsules), sugar-coated tablets, granules, pellets, powders, emulsions, suspensions, aerosols or solutions.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperidoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia power inhalers, nebulizers], nasal drops, solutions, sprays; tablets, films/wafers or capsules, to be administered lingually, sublingually or buccaly, suppositories, preparations for the eyes and the ears, eye baths, ocular insert, ear drops, ear powders, ear-rinses, ear tampons, vaginal capsules, aqueous suspensions (lotions, mixturae agitandae), lipophilic suspensions, ointments, creams, transdermal therapeutic systems (such as, for example, patches), milk, pastes, foams, dusting powders, implants or stents.

Compound A can be converted into the stated administration forms. This can be effected in a manner known per se by mixing with inert, non-toxic, pharmaceutically suitable adjuvants. These adjuvants include, inter alia,
fillers and excipients (for example cellulose, microcrystalline cellulose, such as, for example, Avicel®, lactose, mannitol, starch, calcium phosphate such as, for example, Di-Cafos®),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat)
solvents (for example water, ethanol, Isopropanol, glycerol, propylene glycol, medium chain-length triglycerides fatty oils, liquid polyethylene glycols, paraffins),
surfactants, emulsifiers, dispersants or wetters (for example sodium dodecyle sulphate, lecithin, phospholipids, fatty alcohols such as, for example, Lanette®, sorbitan fatty acid esters such as, for example, Span®, polyoxyethylene sorbitan fatty acid esters such as, for example, Tween®, polyoxyethylene fatty acid glycerides such as, for example, Cremophor®, polyoxethylene fatty acid esters, polyoxyethylene fatty alcohol ethers, glycerol fatty acid esters, poloxamers such as, for example, Pluronic®),
buffers and also acids and bases (for example phosphates, carbonates, citric acid, acetic acid, hydrochloric acid, sodium hydroxide solution, ammonium carbonate, trometamol, triethanolamine)
isotonicity agents (for example glucose, sodium chloride),
adsorbents (for example highly-disperse silicas)
viscosity-increasing agents, gel formers, thickeners and/or binders (for example polyvinylpyrrolidon, methylcellulose, hydroxypropylmethylcellulose, hydroxypropylcellulose, carboxymethylcellulose-sodium, starch, carbomers, polyacrylic acids such as, for example, Carbopol®, alginates, gelatine),
disintegrants (for example modified starch, carboxymethylcellulose-sodium, sodium starch glycolate such as, for example, Explotab®, cross-linked polyvinylpyrrolidon, croscarmellose-sodium such as, for example, AcDiSol®),
flow regulators, lubricants, glidant and mould release agents (for example magnesium stearate, stearic acid, talc, highly-disperse silicas such as, for example, Aerosil®),
coating materials (for example sugar, shellac) and film formers for films or diffusion membranes which dissolve rapidly or in a modified manner (for example polyvinylpyrrolidones such as, for example, Kollidon®, polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®),
capsule materials (for example gelatine, hydroxypropylmethylcellulose),
synthetic polymers (for example polylactides, polyglycolides, polyacrylates, polymethacrylates such as, for example, Eudragit®, polyvinylpyrrolidones such as, for example, Kollidon®, polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to medicaments which comprise at least one compound according to the invention, conventionally together with one or more inert, non-toxic, pharmaceutically suitable adjuvants, and to their use for the above mentioned purposes.

### Dosage and Treatment Regimen

The dosage and the treatment regimen can and must be varied depending on the carcinoma type and the treatment goal.

The daily dose is generally between 20 mg and 850 mg and can be divided into a plurality of identical or different dosage units, preferably 2 which can be taken simultaneously or according to a certain time schedule.

In particular the daily dose is between 30 mg and 500 mg and can be divided into a plurality of identical or different dosage units, preferably 2 which can be taken simultaneously or according to a certain time schedule.

A preferred daily dose is between 20 mg and 400 mg and can be divided into a plurality of identical or different dosage units, preferably 2 which can be taken simultaneously or according to a certain time schedule.

More particularly, the daily dose is between 40 mg and 300 mg and can be divided into a plurality of identical or different dosage units, preferably 2 which can be taken simultaneously or according to a certain time schedule.

A more preferred daily dose is between 20 mg and 200 mg and can be divided into a plurality of identical or different dosage units, preferably 2 which can be taken simultaneously or according to a certain time schedule.

An even more preferred daily dose is between 50 mg and 180 mg and can be divided into a plurality of identical or different dosage units, preferably 2 which can be taken simultaneously or according to a certain time schedule.

This applies both to monotherapy and to combination therapy with other anti-hyperproliferative, cytostatic or cytotoxic substances, the combination therapy possibly requiring a reduction in dose.

The treatment can be carried out in regularly repeated cycles. Treatment cycles may have varying duration, such as 21 days or 28 days, whereby dosing is given continuously, or intermittently. Preferred is a cycle length of 28 days, whereby dosing is given continuously, or intermittently.

Continuous schedules involve daily dosing, for example, 21 daily doses in a 21 day cycle, or 28 daily doses in a 28 day cycle. A preferred continuous schedule is 28 daily doses in a 28 daily cycle.

Intermittent schedules involve a period of treatment followed by a period of non-treatment, for example in a cycle of 21 days, or a cycle of 28 days. A preferred cycle duration for an intermittent schedule is 28 days.

The period of treatment may be repeated more than once in a given treatment cycle.

The period of treatment may be for example 1 to 21 days, more preferably 3 to 14 days.

An even more preferred intermittent schedule involves treatment for 3 days followed by non-treatment for 4 days, repeated every week in such a way that a 28 day treatment cycle is completed.

Treatment is successful when there is at least disease stabilization and the adverse effects occur to an extent which is easily treatable, but at least easily acceptable. Thus the number of cycles of treatment applied may vary from patient to patient, according to treatment response and tolerability.

Treatment is successful when there is at least disease stabilization and the adverse effects occur to an extent which is easily treatable, but at least easily acceptable.

Compound A can be used on its own or, if required, in combination with one or more other pharmacologically effective substances, provided said combination does not lead to undesired and unacceptable adverse effects. The present invention therefore further provides drugs containing compound A according to the invention and one or more further active ingredients, in particular for treating and/or preventing the above-mentioned diseases.

For example, compound A can be combined with known anti-hyperproliferative, cytostatic or cytotoxic substances for treating cancers. The combination compound A according to the invention with other substances in use for cancer therapy or else with radiotherapy is especially advisable.

Examples of suitable active ingredients for combination purposes include: abraxane, afinitor, aldesleukin, alendronic acid, alfaferone, alitretinoin, allopurinol, aloprim, aloxi, altretamine, aminoglutethimide, amifostine, amrubicin, amsacrine, anastrozole, anzemet, aranesp, arglabin, arsenic trioxide, aromasin, 5-azacytidine, azathioprine, BCG or tice-BCG, bestatin, betamethasone acetate, betamethasone sodium phosphate, bexarotene, bleomycin sulphate, broxuridine, bortezomib, busulfan, calcitonin, campath, capecitabine, carboplatin, casodex, cefesone, celmoleukin, cerubidine, chlorambucil, cisplatin, cladribine, clodronic acid, cyclophosphamide, cytarabine, dacarbazine, dactinomycin, daunoxome, decadron, decadron phosphate, delestrogen, denileukin diftitox, depo-medrol, deslorelin, dexrazoxane, diethylstilbestrol, diflucan, docetaxel, doxifluridine, doxorubicin, dronabinol, DW-166HC, eligard, elitek, ellence, emend, epirubicin, epoetin alfa, epogen, eptaplatin, ergamisol, estrace, estradiol, estramustine sodium phosphate, ethinyl estradiol, ethyol, etidronic acid, etopophos, etoposide, fadrozole, fareston, filgrastim, finasteride, fligrastim, floxuridine, fluconazole, fludarabine, 5-fluorodeoxyuridine monophosphate, 5-fluorouracil (5-FU), fluoxymesterone, flutamide, formestane, fosteabine, fotemustine, fulvestrant, gammagard, gemcitabine, gemtuzumab, gleevec, gliadel, goserelin, granisetron hydrochloride, histrelin, hycamtin, hydrocortone, erythro-hydroxynonyladenine, hydroxyurea, ibritumomab tiuxetan, idarubicin, ifosfamide, interferon alpha, interferon alpha 2, interferon alpha 2α, interferon alpha 2β, interferon alpha n1, interferon alpha n3, interferon beta, interferon gamma 1α, interleukin 2, intron A, iressa, irinotecan, kytril, lapatinib, lentinan sulphate, letrozole, leucovorin, leuprolide, leuprolide acetate, levamisole, levofolinic acid calcium salt, levothroid, levoxyl, lomustine, lonidamine, marinol, mechlorethamine, mecobalamin, medroxyprogesterone acetate, megestrol acetate, melphalan, menest, 6-mercaptopurine, mesna, methotrexate, metvix, miltefosine, minocycline, mitomycin C, mitotane, mitoxantrone, modrenal, myocet, nedaplatin, neulasta, neumega, neupogen, nilutamide, nolvadex, NSC-631570, OCT-43, octreotide, ondansetron hydrochloride, orapred, oxaliplatin, paclitaxel, pediapred, pegaspargase, pegasys, pentostatin, picibanil, pilocarpine hydrochloride, pirarubicin, plicamycin, porfimer sodium, prednimustine, prednisolone, prednisone, premarin, procarbazine, procrit, raltitrexed, RDEA119, rebif, rhenium-186 etidronate, rituximab, roferon-A, romurtide, salagen, sandostatin, sargramostim, semustine, sizofiran, sobuzoxane, solu-medrol, streptozocin, strontium-89 chloride, synthroid, tamoxifen, tamsulosin, tasonermin, tastolactone, taxotere, teceleukin, temozolomide, teniposide, testosterone propionate, testred, thioguanine, thiotepa, thyrotropin, tiludronic acid, topotecan, toremifene, tositumomab, trastuzumab, treosulfan, tretinoin, trexall, trimethylmelamine, trimetrexate, triptorelin acetate, triptorelin pamoate, UFT, uridine, valrubicin, vesnarinone, vinblastine, vincristine, vindesine, vinorelbine, virulizin, zinecard, zinostatin stimalamer, zofran; ABI-007, acolbifene, actimmune, affinitak, aminopterin, arzoxifene, asoprisnil, atamestane, atrasentan, BAY 43-9006 (sorafenib), avastin, CCI-779, CDC-501, celebrex, cetuximab, crisnatol, cyproterone acetate, decitabine, DN-101, doxorubicin MTC, dSLIM, dutasteride, edotecarin, eflornithine, exatecan, fenretinide, histamine dihydrochloride, histrelin hydrogel implant, holmium-166 DOTMP, ibandronic acid, interferon gamma, intron-PEG, ixabepilone, keyhole limpet hemocyanin, L-651582, lanreotide, lasofoxifene, libra, lonafarnib, miproxifen, minodronate, MS-209, liposomal MTP-PE, MX-6, nafarelin, nemorubicin, neovastat, nolatrexed, oblimersen, onco-TCS, osidem, paclitaxel polyglutamate, pamidronate disodium, PN-401, QS-21, quazepam, R-1549, raloxifene, ranpirnase, 13-cis-retinoic acid, satraplatin, seocalcitol, T-138067, tarceva, taxoprexin, thymosin alpha 1, tiazofurin, tipifarnib, tirapazamine, TLK-286, toremifene, transMID-107R, valspodar, vapreotide, vatalanib, verteporfin, vinflunine, Z-100, zoledronic acid, and also combinations thereof.

In a preferred embodiment, compound A of the present invention can be combined with the following active ingredients:

131I-chTNT, abarelix, abiraterone, aclarubicin, aldesleukin, alemtuzumab, alitretinoin, altretamine, aminoglutethimide, amrubicin, amsacrine, anastrozole, arglabin, arsenic trioxide, asparaginase, azacitidine, basiliximab, BAY 80-6946, belotecan, bendamustine, bevacizumab, bexarotene, bicalutamide, bisantrene, bleomycin, bortezomib, buserelin, busulfan, cabazitaxel, calcium folinate, calcium levofolinate, capecitabine, carboplatin, carmofur, carmustine, catumaxomab, celecoxib, celmoleukin, cetuximab, chlorambucil, chlormadinone, chlormethine, cisplatin, cladribine, clodronic acid, clofarabine, crisantaspase, cyclophosphamide, cyproterone, cytarabine, dacarbazine, dactinomycin, darbepoetin alfa, dasatinib, daunorubicin, decitabine, degarelix, denileukin diftitox, denosumab, deslorelin, dibrospidium chloride, docetaxel, doxifluridine, doxorubicin, doxorubicin+estrone, eculizumab, edrecolomab, elliptinium acetate, eltrombopag, endostatin, enocitabine, epirubicin, epitiostanol, epoetin alfa, epoetin beta, eptaplatin, eribulin, erlotinib, estradiol, estramustine, etoposide, everolimus, exemestane, fadrozole, filgrastim, fludarabine, fluorouracil, flutamide, formestane, fotemustine, fulvestrant, gallium nitrate, ganirelix, gefitinib, gemcitabine, gemtuzumab, glutoxim, goserelin, histamine dihydrochloride, histrelin, hydroxycarbamide, I-125 seeds, ibandronic acid, ibritumomab tiuxetan, idarubicin, ifosfamide, imatinib, imiquimod, improsulfan, interferon alpha, interferon beta, interferon gamma, ipilimumab, irinotecan, ixabepilone, lanreotide, lapatinib, lenalidomide, lenograstim, lentinan, letrozole, leuprorelin, levamisole, lisuride, lobaplatin, lomustine, lonidamine, masoprocol, medroxyprogesterone, megestrol, melphalan, mepitiostane, mercaptopurine, methotrexate, methoxsalen, methyl aminolevulinate, methyltestosterone, mifamurtide, miltefosine, miriplatin, mitobronitol, mitoguazone, mitolactol, mitomycin, mitotane, mitoxantrone, nedaplatin, nelarabine, nilotinib, nilutamide, nimotuzumab, nimustine, nitracrine, ofatumumab, omeprazole, oprelvekin, oxaliplatin, p53 gene therapy, paclitaxel, palifermin, palladium-103 seed, pamidronic acid, panitumumab, pazopanib, pegaspargase, PEG-epoetin beta (methoxy-PEG-epoetin beta), pegfilgrastim, peginterferon alfa 2b, pemetrexed, pentazocine, pentostatin, peplomycin, perfosfamide, picibanil, pirarubicin, plerixafor, plicamycin, poliglusam, polyestradiol phosphate, polysaccharide-K, porfimer sodium, pralatrexate, prednimustine, procarbazine, quinagolide, radium-223 chloride, raloxifene, raltitrexed, ranimustine, razoxane, refametinib, regorafenib, risedronic acid, rituximab, romidepsin, romiplostim, sargramostim, sipuleucel-T, sizofiran, sobuzoxane, sodium glycididazole, sorafenib, streptozocin, sunitinib, talaporfin, tamibarotene, tamoxifen, tasonermin, teceleukin, tegafur, tegafur+gimeracil+oteracil, temoporfin, temozolomide, temsirolimus, teniposide, testosterone, tetrofosmin, thalidomide, thiotepa, thymalfasin, tioguanine, tocilizumab, topotecan, toremifene, tositumomab, trabectedin, trastuzumab, treosulfan, tretinoin, trilostane, triptorelin, trofosfamide, tryptophan, ubenimex, valrubicin, vandetanib, vapreotide, vemurafenib, vinblastine, vincristine, vindesine, vinflunine, vinorelbine, vorinostat, vorozole, yttrium-90 glass microspheres, zinostatin, zinostatin stimalamer, zoledronic acid, zorubicin.

Promisingly, compound A can also be combined with biological therapeutics such as antibodies (e.g. avastin, rituxan, erbitux, herceptin, cetuximab) and recombinant proteins.

Compound A can also achieve positive effects in combination with other therapies directed against angiogenesis, such as, for example, with avastin, axitinib, regorafenib, recentin, sorafenib or sunitinib. Combinations with inhibitors of the proteasome and of mTOR and also antihormones and steroidal metabolic enzyme inhibitors are especially useful because of their favourable profile of adverse effects.

In general, the combination of compound A with other cytostatic or cytotoxic agents makes it possible to pursue the following goals:
improved efficacy in slowing the growth of a tumour, in reducing its size or even in completely eliminating it in comparison with treatment using an individual active ingredient;
the possibility of employing the chemotherapeutics used in a lower dosage than in the case of monotherapy;
the possibility of a more tolerable therapy with fewer adverse effects in comparison with individual administration;
the possibility of treating a broader spectrum of tumour diseases;
achieving a higher response rate to the therapy;
longer patient survival time in comparison with current standard therapy.

Furthermore, compound A according to the invention can also be used in connection with radiotherapy and/or a surgical intervention.

### EXAMPLES

### 1. Preparation of Compound A

Compound A′ was prepared according to the procedure described in example 4 of WO2012/160034.

### 2. Proliferation Assay

TABLE 1
List of the cell lines investigated and
results of the proliferation assays.

| | Example | Compound A′ |
|---|---|---|
| Tumor indication | Cell line | IC₅₀ [nmol/l] |
| Acute leukemia | HL-60 | 1125 |
| Acute myeloid leukemia, 11q23 positive | MOLM-13 | 320 |
| Acute myeloid leukemia, 11q23 positive | MV4-11 | 386 |
| Acute myeloid leukemia, 11q23 positive | NOMO-1 | 328 |
| Acute myeloid leukemia | KG-1 | 670 |
| Acute myeloid leukemia | PL-21 | 192 |
| Acute myeloid leukemia | OCI-AML-2 | 323 |

^{#}After 72 hours of incubation with the substance

3. In-vivo experiments

The aim of the present experiments was to assess the in vivo efficacy and tolerability of Compound A′ in monotherapy in four leukemia models subcutaneously implanted in NOD/SCID mice.

### 3.1 Acronyms and Abbreviations

TABLE 2
Acronyms and Abbreviations

| | |
|---|---|
| BW | Body weight |
| BW₀ | Individual body weight at day 0 |
| BWₓ | Individual body weight at day X |
| BWL | Body weight loss |
| MLL | Mixed lineage leukemia |
| n/a | Not applicable |
| NOD/SCID | Non obese diabetic/severe combined immunodeficiency |
| n.s. | Statistically not significant |
| p.o. | Per os, orally |
| s. | Statistically significant |
| T/C | Relative Tumor versus control value |
| RTV | Relative tumor volume |

### 3.2 Design

The experiments included four in vivo efficacy experiments with female NOD/SCID mice bearing subcutaneous leukemia xenografts. Compound A′ was assessed at one dose level in monotherapy. Anti-tumor activity and tolerability of all groups were assessed using the vehicle control group as a reference.

| | | Total Daily | Schedule | | |
|---|---|---|---|---|---|
| Group | | Dose | [Dosing | Appl. | No. of |
| ID | Therapy | [mg/kg/day] | days] | Route | Animals |
| 1 | Vehicle | 10 mL/kg/day | 0-25 | p.o. | 8-10 |
| 2 | Compound A′ | 25 | 0-25 | p.o. | 8-10 |

### 3.3 Experimental Procedures

### 3.3.1. Specific Animal Information

Mouse strain, sex: NOD/SCID, female
Animals supplied by: Harlan
Total number of mice
Efficacy test (implanted/randomized): 100/74
Approximate age at implantation: 5-7 weeks
Approximate age at randomization: 6-9 weeks

### Housing Conditions

The animals were housed in individually ventilated cages. The animals were monitored twice daily. All materials were autoclaved prior to use. Food and water were provided ad libitum.

### 3.3.2 Tumor Information

### 3.3.2.1 Characterization of Test Tumors

The tumor models used in this study were derived from commercially available cell lines. The three leukemia xenograft models MV4-11, NOMO-1 and THP-1 carried an 11q23 translocation (also referred to by other terms including 11q23 rearrangement, 11q rearrangements and the MLL rearrangement), whereas model HL-60 did not exhibit such an alteration.

### 3.3.2.2 Tumor Implantation

Leukemia cells were subcutaneously injected as cell suspensions into one flank of immunodeficient NOD/SCID mice under isofluorane anaesthesia (5*10⁶ cells per injection). In the case of THP-1 cells, matrigel was used to increase implantation efficacy. For this, 1×10⁸ leukemia cells were mixed with matrigel and an appropriate volume of the solution containing 5*10⁶ cells was injected as described above.

### 3.3.3 Randomization

Animals and tumor implants were monitored daily until the maximum number of implants showed clear signs of beginning solid tumor growth. At randomization, the volume of growing tumors was initially determined. Animals bearing one tumor of a volume of 50-250 mm³, preferably 80-200 mm³, were distributed in experimental groups according to the study protocol, considering a comparable median and mean of group tumor volume of approximately 100-120 mm³. The result of the randomization was documented and maintained with the experimental data. Animals not randomized were euthanized. The day of randomization is designated as day 0 of an experiment.

### 3.3.4. Test Reagents

Vehicle: 80% (m/V) PEG400 in water for injection
Compound A′: preparation of a dosing solution (2.5 mg/ml) once weekly by diluting the Compound A′ powder at 0.25% (w/v) in vehicle; storage of the dosing solution at 4° C.; dosing volume 10 ml/kg

### 3.3.5. Observations and Calculations

### 3.3.5.1 Mortality

Mortality checks were conducted daily during routine monitoring.

### 3.3.5.2 Body Weight

Mice were weighed twice a week. Relative body weights of individual mice in % were calculated by dividing the individual body weight on day X (BW_{X}) by the individual body weight on day 0 (BW₀) multiplied by 100 according to the formula:

$$Relative Body Weight ( {Day}_{x} ) [ \% ] = \frac{{BW}_{X}}{{BW}_{0}} \times 100$$

Group median relative body weights were calculated as well, considering only the weights of mice that were alive on the day in question.

### 3.3.5.3 Tumor Volume

The tumor volumes were determined by two-dimensional measurement with a caliper on the day of randomization (day 0) and then twice weekly (i.e. on the same days on which mice were weighed). Tumor volumes were calculated according to the formulas:

Tumor volume=(a×b²)×0.5

where a represents the largest and b the perpendicular tumor diameter.

### 3.3.5.4 Anti-Tumor Activity

Anti-tumor activity was evaluated as maximum tumor volume inhibition versus the vehicle control group. Data evaluation was performed using Oncotest proprietary software.

### 3.3.5.5 Tumor Inhibition, Test/Control Value in %

Tumor inhibition for a particular day (T/C in %) was calculated from the ratio of the median RTV values of test versus control groups multiplied by 100.

$$T / C ( {Day}_{x} ) [ \% ] = \frac{Median relative tumor volume of the test group on {Day}_{x}}{Median relative tumor volume of the control group on {Day}_{x}} = 100$$

The minimum (or optimum) T/C % value recorded for a particular test group during an experiment represents the maximum anti-tumor activity for the respective treatment. T/C values were calculated if at least 50% of the randomized animals in a group were alive on the day in question.

### 3.3.5.6 Efficacy Criteria

Group optimum T/C values (in %) were used for activity rating as follows:

TABLE 3
Efficacy criteria

| | | |
|---|---|---|
| − | Inactive | T/C ≧ 65% |
| +/− | Borderline activity | 50% ≦ T/C < 65% |
| + | Moderate activity | 25% ≦ T/C < 50% |
| ++ | High activity | 10% ≦ T/C < 25% |
| +++ | Very high activity | 5% ≦ T/C < 10% |
| ++++ | Complete remission | T/C < 5% |

### 3.4 Results

### 3.4.1 Anti-Tumor Efficacy of Compound A′ in Xenograft-Bearing Mice

Compound A′ was assessed at one dose level in four leukemia models implanted subcutaneously in NOD/SCID mice. The three leukemia xenograft models MV4-11, NOMO-1 and THP-1 carried an 11q23 translocation (also referred to by other terms including 11q23 rearrangement, 11q rearrangements and the MLL rearrangement), whereas model HL-60 did not exhibit such an alteration High anti-tumor activity was observed in MV4-11, NOMO-1 and THP-1 with minimum T/C values ranging from 11.9% to 21.0%, while borderline anti-tumor activity was observed in HL-60 (min. T/C value: 63.9%).

Tumor growth of MV4-11, NOMO-1 and THP-1 was significantly reduced by Compound A′ treatment as compared to the respective vehicle control groups, as determined by the non-parametric Mann-Whitney-Wilcoxon U-test.

TABLE 4
Summary of Anti-tumor efficacy of Compound A′

| | | | | | |
|---|---|---|---|---|---|
| Group | | Dose Level | | Min. T/C | Activity |
| ID | Therapy | [mg/kg/day] | Schedule | [%] (Day) | Rating |

HL-60

| | | | | | |
|---|---|---|---|---|---|
| 1 | Vehicle | 10 ml/kg/day | 0-6, 11-17 | n/a | n/a |
| 2 | Compound A′ | 25 | 0-6, 11-17 | 63.9 (14) | +/− |

MV4-11

| | | | | | |
|---|---|---|---|---|---|
| 1 | Vehicle | 10 ml/kg/day | 0-25 | n/a | n/a |
| 2 | Compound A′ | 25 | 0-25 | 11.9 (14) | ++ |

NOMO-1

| | | | | | |
|---|---|---|---|---|---|
| 1 | Vehicle | 10 ml/kg/day | 0-17 | n/a | n/a |
| 2 | Compound A′ | 25 | 0-25 | 19.7 (14) | ++ |

THP-1

| | | | | | |
|---|---|---|---|---|---|
| 1 | Vehicle | 10 ml/kg/day | 0-20 | n/a | n/a |
| 2 | Compound A′ | 25 | 0-25 | 21.0 (14) | + |

* Vehicle. 80% PEG400 in water for injection

In conclusion, these data indicate significant and meaningful anti-tumor activity of Compound A′ in patients with leukemias, especially in the acute myeloid leukemia (AML) type. AML patients carrying an 11q23 translocation (also referred to by other terms including 11q23 rearrangement, 11q rearrangements and the MLL rearrangement) are expected to show an even increased anti-tumor efficacy upon treatment with Compound A′.

### 3.4.2. Survival and Body Weight Changes

No or minor group median BWLs up to 0.4% were observed in tumor models NOMO-1 and THP-1, while moderate and substantial group median BWLs of 10.9% and 9.1% were observed in HL-60 and MV4-11, respectively. The survival rates ranged from 67% to 100%.

In conclusion, Compound A′ showed an acceptable tolerability profile in leukemia xenografts bearing mice.

### 3.5. Summary and Conclusion

The in vivo efficacy and tolerability of BHC's investigational compound Compound A′ was assessed in monotherapy in four subcutaneously implanted cell line derived leukemia xenograft models. The three leukemia xenograft models MV4-11, NOMO-1 and THP-1 carried an 11q23 translocation (also referred to by other terms including 11q23 rearrangement, 11q rearrangements and the MLL rearrangement), whereas model HL-60 did not exhibit such an alteration.

All leukemia models were implanted by subcutaneous injection of the corresponding cell line into female NOD/SCID mice. Compound A′ was administered orally at one dose level (25 mg/kg/day) in monotherapy once daily and treatment started once subcutaneous tumors were established. A vehicle-treated control group was included in each experiment. Group sizes were eight to ten mice per group. Anti-tumor activity (tumor growth inhibition) and tolerability of all groups were assessed using the vehicle control group as a reference.

High anti-tumor activity was observed in MV4-11, NOMO-1 and THP-1 with minimum T/C values ranging from 11.9% to 21.0%, while borderline anti-tumor activity was observed in HL-60 (min. T/C value: 63.9%). Tumor growth in the three former models was significantly attenuated by Compound A′ treatment as compared to the respective vehicle control groups (Mann-Whitney-Wilcoxon U-test). No or minor group median BWLs up to 0.4% were observed in tumor models NOMO-1 and THP-1, while moderate and substantial group median BWLs up to 10.9% were observed in HL-60 and MV4-11.

In conclusion, these data indicate significant and meaningful anti-tumor activity of Compound A′ in patients with acute leukemias, especially in the acute myeloid leukemia (AML) type. AML patients carrying an 11q23 translocation (also referred to by other terms including 11q23 rearrangement, 11q rearrangements and the MLL rearrangement) are expected to show an even increased anti-tumor efficacy upon treatment with Compound A′.

1-6. (canceled)

7: A method for identifying a patient disposed to respond favorably to a CDK9-inhibitor for treating acute myeloid leukemia,
wherein the CDK9-inhibitor is a compound 4-(4-Fluoro-2-methoxyphenyl)-N-{3-[(S-methylsulfonimidoyl)methyl]phenyl}-1,3,5-triazin-2-amine according to formula I 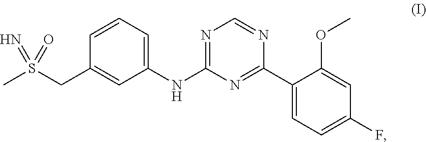 [SMILES: COc1cc(F)ccc1-c1ncnc(Nc2cccc(CS(C)(=N)=O)c2)n1]
or a physiologically acceptable salt thereof, and
wherein the method comprises the detection of a 11q23 translocation in tumor cells in a tissue sample from the patient.

8: The method according to claim 7, wherein the 11q23 translocation is detected by a cytogenetic analysis.

9-18. (canceled)

19: A method for treating an acute myeloid leukemia comprising the steps of
a) assaying a tumor sample from the patient; and
b) determining if 11q23 is translocated; and
c) administering a therapeutically effective amount of
4-(4-Fluoro-2-methoxyphenyl)-N-{3-[(S-methylsulfonimidoyl)methyl]phenyl}-1,3,5-triazin-2-amine according to formula I 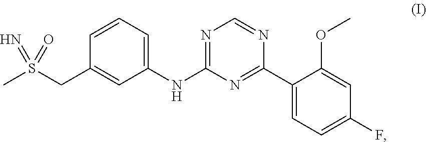 [SMILES: COc1cc(F)ccc1-c1ncnc(Nc2cccc(CS(C)(=N)=O)c2)n1]
or a physiologically acceptable salt thereof, to the patient,
if 11q23 is translocated as defined in step b.

20. (canceled)

21: The method according to claim 19, wherein (+)-4-(4-Fluoro-2-methoxyphenyl)-N-{3-[(S-methylsulfonimidoyl)methyl]phenyl}-1,3,5-triazin-2-amine is used.

22-23. (canceled)

24: The method according to claim 19, wherein the translocation is detected by a cytogenetic analysis.

25: A method for treatment and/or prophylaxis of leukemia comprising administering a therapeutically effective amount of a pharmaceutical combination comprising
4-(4-Fluoro-2-methoxyphenyl)-N-{3-[(S-methylsulfonimidoyl)methyl]phenyl}-1,3,5-triazin-2-amine according to formula I 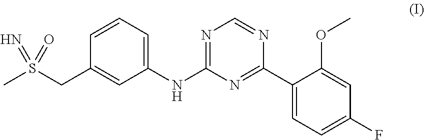 [SMILES: COc1cc(F)ccc1-c1ncnc(Nc2cccc(CS(C)(=N)=O)c2)n1]
or a physiologically acceptable salt thereof, and at least one or more further active ingredients to a subject in need thereof.

26: A method for treatment and/or prophylaxis of leukemia comprising administering a therapeutically effective amount of a pharmaceutical composition comprising
4-(4-Fluoro-2-methoxyphenyl)-N-{3-[(S-methylsulfonimidoyl)methyl]phenyl}-1,3,5-triazin-2-amine according to formula I 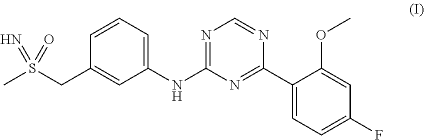 [SMILES: COc1cc(F)ccc1-c1ncnc(Nc2cccc(CS(C)(=N)=O)c2)n1]
or a physiologically acceptable salt thereof, and an inert, nontoxic, pharmaceutically suitable adjuvant to a subject in need thereof.

27: The method according to claim 25, wherein the pharmaceutical combination comprises (+)-4-(4-Fluoro-2-methoxyphenyl)-N-{3-[(S-methylsulfonimidoyl)methyl]phenyl}-1,3,5-triazin-2-amine.

28: The method according to claim 25, wherein the leukemia is an acute myeloid leukemia.

29: The method according to claim 28, wherein the subject in need thereof is one for whom a 11q23 translocation has been detected in a tissue sample containing tumor cells from the subject.

30: The method according to claim 29, wherein the translocation is detected by a cytogenetic analysis.

31. (canceled)

32: A method for treatment and/or prophylaxis of leukemia comprising administering a therapeutically effective amount of 4-(4-Fluoro-2-methoxyphenyl)-N-{3-[(S-methylsulfonimidoyl)methyl]phenyl}-1,3,5-triazin-2-amine according to formula I 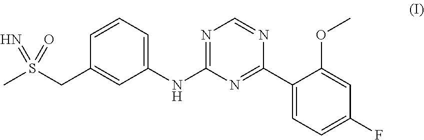 [SMILES: COc1cc(F)ccc1-c1ncnc(Nc2cccc(CS(C)(=N)=O)c2)n1]
or a physiologically acceptable salt thereof, to a subject in need thereof.

33: The method according to claim 32, wherein (+)-4-(4-Fluoro-2-methoxyphenyl)-N-{3-[(S-methylsulfonimidoyl)methyl]phenyl}-1,3,5-triazin-2-amine is used.

34: The method according to claim 32, wherein the leukemia is an acute myeloid leukemia.

35: The method according to claim 34, wherein the subject in need thereof is one for whom a 11q23 translocation has been detected in a tissue sample containing tumor cells from the subject.

36: The method according to claim 35, wherein the translocation is detected by a cytogenetic analysis.

37: The method according to claim 26, wherein the pharmaceutical composition comprises
(+)-4-(4-Fluoro-2-methoxyphenyl)-N-{3-[(S-methylsulfonimidoyl)methyl]phenyl}-1,3,5-triazin-2-amine.

38: The method according to claim 26, wherein the leukemia is an acute myeloid leukemia.

39: The method according to claim 38, wherein the subject in need thereof is one for whom a 11q23 translocation has been detected in a tissue sample containing tumor cells from the subject.

40: The method according to claim 39, wherein the translocation is detected by a cytogenetic analysis.
